(19) United States
(12) Patent Application Publication
(10) Pub. No.: US 2015/0118385 A1
(43) Pub. Date: Apr. 30, 2015

(54) METHOD FOR INCREASING PHOTOSYNTHETIC CARBON FIXATION IN RICE

(71) Applicant: Bayer BioScience N.V., Gent (BE)

(72) Inventors: Rashad Kebeish, Hannover (DE); Fritz Kreuzaler, Aachen (DE); Michael Metzlaff, Tervuren (BE); Markus Niessen, Hannover (DE); Christoph Peterhaensel, Hannover (DE); Jeroen Van Rie, Eeklo (BE)

(73) Assignee: Bayer BioScience N.V., Gent (BE)

(21) Appl. No.: 14/576,874

(22) Filed: Dec. 19, 2014

Related U.S. Application Data
(62) Division of application No. 13/056,708, filed on Apr. 25, 2011.

(30) Foreign Application Priority Data
Aug. 1, 2008 (EP) 08161682.3

(51) Int. Cl. C12N 15/82; A23L 1/182; A23L 1/10; C12N 9/04

(52) U.S. Cl. CPC C12N 15/8225; C12N 15/8261; C12N 9/0006; A23L 1/182; A23L 1/1041; C12Y 101/99014; A23V 2002/00
USPC 426/622; 800/290; 800/278; 800/320.2; 800/287; 426/627

(57) ABSTRACT

The invention relates to a method for stimulating the growth of the plants and/or improving the biomass production and/or increasing the carbon fixation by the plant comprising introducing into a rice plant cell, rice plant tissue or rice plant one or more nucleic acids, wherein the introduction of the nucleic acid(s) results inside the chloroplast of a de novo expression of one or more polypeptides having the enzymatic activity of a glycolate dehydrogenase.

### REFERENCE TO RELATED APPLICATIONS

The present application is a divisional application of application Ser. No. 13/056,708, filed Apr. 25, 2011, which is a U.S. National Stage Application of PCT Application No. PCT/EP2009/059843 filed Jul. 30, 2009, which claims priority to European application No. 08161682.3 filed Aug. 1, 2008. The entire contents of each of these applications are hereby incorporated by reference herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 13388_{—}10_Sequence_Listing. The size of the text file is 106 KB, and the text file was created on Dec. 19, 2014.

Rice (Oriza sativa) is the most important cereal grown globally and the major staple food for about half of the world population. With the growing world population and the increasing pressure over available arable land worldwide, crop productivity in the field needs to be constantly improved. There is therefore a constant need for new solutions contributing to the increase of crop productivity, and rice, as the most important cereal, is one major target crop for such solutions.

Crop productivity is influenced by many factors, among which are, on the one hand factors influencing the capacity of the plant to produce biomass (photosynthesis, nutrient and water uptake), and on the other hand factors influencing the capacity of the plant to resist certain stresses, like biotic stresses (insects, fungi, viruses . . . ) or abiotic stresses (drought, salinity . . . ).

One important factor influencing the production of biomass is photosynthesis. Photosynthesis is the mechanism through which plants capture atmospheric carbon dioxide and transform it into sugar, which is then incorporated into plant tissues, thereby creating biomass.

Most plants have a photosynthetic mechanism in which the chloroplastic enzyme RuBisCo (Ribulose-1,5-Bisphosphate Carboxylase/Oxygenase) is the main enzyme capturing carbon dioxide and transforming it into sugar. Those plants are called C3 plants, and rice is a C3 plant. One known problem in the photosynthetic mechanism of C3 plants is that the efficiency of carbon fixation is not optimal in certain environmental conditions where part of the fixed carbon is lost through the alternative activity of RuBisCo called oxygenation.

RuBisCO is able to catalyze both the carboxylation and oxygenation of ribulose-1,5-bisphosphate. The balance between these two activities depends mainly on the CO₂/O₂ ratio in the leaves, which may change following the plant's reaction to certain environmental conditions. Each carboxylation reaction produces two molecules of phosphoglycerate that enter the Calvin cycle, ultimately to form starch and sucrose and to regenerate ribulose-1,5-bisphosphate. The oxygenation reaction produces single molecules of phosphoglycerate and phosphoglycolate. The latter is recycled into phosphoglycerate by photorespiration (Leegood R. C. et al, 1995). One molecule of CO₂ is released for every two molecules of phosphoglycolate produced, resulting in a net loss of fixed carbon that ultimately reduces the production of sugars and biomass. Ammonia is also lost in this reaction, and needs to be refixed through energy consuming reactions in the chloroplast.

Overcoming photorespiration has been reported as a target for raising the maximum efficiency of photosynthesis and enhancing its productivity (Zhu et al., 2008) and several attempts have been described so far to reduce the loss of carbon in plants and therefore to increase the production of sugars and biomass. Some promising results have been obtained in some plant species, but so far, no positive results have been reported in rice.

Kebeish et al. reported that the photorespiratory losses in Arabidopsis thaliana can be alleviated by introducing into chloroplasts a bacterial pathway for the catabolism of the photorespiratory substrate, glycolate (WO 03/100066; Kebeish R. et al., 2007). The authors first targeted the three subunits of Escherichia coli glycolate dehydrogenase to Arabidopsis thaliana chloroplasts and then introduced the Escherichia coli glyoxylate carboligase and Escherichia coli tartronic semialdehyde reductase to complete the pathway that converts glycolate to glycerate in parallel with the endogenous photorespiratory pathway. This step-wise nuclear transformation with the five Escherichia coli genes leads to Arabidopsis plants in which chloroplastic glycolate is converted directly to glycerate. These transgenic plants grew faster, produced more shoot and root biomass, and contained more soluble sugars. An effect was also visible but to a lesser extent in Arabidopsis plants that overexpressed only the three subunits of the glycolate dehydrogenase.

Another strategy is to transfer C₄- or C₄-like pathways or components of this pathway to C₃ plants.

In 1996, Gehlen J. et al. reported a change in photosynthetic characteristics at optimal temperatures in transgenic potato expressing a bacterial PEPC (phosphoenolpyruvate carboxylase) gene from C. glutamicum.

This approach has been applied to rice in 1999 by Ku et al, using the maize PEPC. Nevertheless, in transgenic rice plants, the rate of CO₂ assimilation was not altered significantly, and there was only a weak impact on plant physiology and growth performance, although an increase in PEPC activity levels up to 100-fold was detected (Matsuoka et al., 2001; see also EP-A 0 874 056).

Another study reported that overexpression of a phosphoenolpyruvate carboxykinase (PCK) from Urochloa panicoides targeted to rice chloroplasts resulted in the induction of endogenous PEPC and the establishment of a C₄-like cycle within a single cell. However, no enhanced growth parameters were observed (Suzuki et al., 2000; see also WO 98/35030). Recently (2008), Y. Taniguchi et al. introduced the C₄-like pathway of Hydrilla verticillata into the mesophyll cell of a rice plant. Different transgenic rice plants overproducing independently or in combination four C₄ enzymes, namely phosphoenolpyruvate carboxylase, orthophosphate dikinase, NADP-malic enzyme, and NADP-malate dehydrogenase, were produced. It was found that overproduction of all four enzymes in combination slightly improved photosynthesis, but at the same time caused slight but reproducible stunting of transgenic plant growth.

There is therefore still a need for an efficient method for increasing the carbon fixation in rice, which would stimulate the growth of the plant and/or improve biomass and/or seed production.

The present invention relates to a method for increasing biomass production and/or seed production and/or carbon fixation in rice plants comprising introducing into the genome of a rice plant cell one or more nucleic acids encoding one or more polypeptides having the enzymatic activity of a glycolate dehydrogenase, wherein said introduction of said one or more nucleic acids results in a de novo expression of one or more polypeptides having the enzymatic activity of a glycolate dehydrogenase and wherein said one or more polypeptides are localized in chloroplasts of the rice plant produced.

In the context of the invention, biomass is the quantity of matter produced by individual plants, or by surface area on which the plants are grown. Several parameters may be measured in order to determine the increase of biomass production. Examples of such parameters are the height of the plant, surface of the leave blade, shoot dry weight, root dry weight, seed number, seed weight, seed size, . . . . In that respect, seed production, or seed yield, is one specific indicator of biomass. Seed production or seed yield can be measured per individual plant or per surface area where the plants are grown. These parameters are generally measured after a determined period of growth in soil or at a specific step of growth, for example at the end of the vegetative period, and compared between plants transformed with the one or more nucleic acids according to the invention and plants not transformed with such one or more nucleic acids.

The increase of carbon fixation by the plant can be determined by measuring gas exchange and chlorophyll fluorescence parameters. A convenient methodology, using the LI-6400 system (Li-Cor) and the software supplied by the manufacturer, is described in R. Kebeish et al., 2007, and is incorporated herein by reference.

The nucleic acids involved in the method of the invention encode(s) one or more polypeptides having the enzymatic activity of a glycolate dehydrogenase.

The enzymatic activity of glycolate dehydrogenases can be defined by the oxidation of glycolate to form glyoxylate using organic cofactors, whereas glycolate oxidases, present for example in plant peroxisomes, use molecular oxygen as a cofactor and release hydrogen peroxide.

Such clear distinction between glycolate dehydrogenases and glycolate oxidases based on the nature of the cofactors have not always be done, and as an example the E. coli glycolate dehydrogenase encoded by the gcl operon was previously named glycolate oxidase (Bari et al., 2004).

The glycolate dehydrogenase activity can be assayed according to Lord J. M. 1972, using the technology described in example 4 of the present application.

Alternatively, complementation analysis with mutants of E. coli deficient in the three subunits forming active endogenous glycolate dehydrogenase may be performed. These mutants of E. coli are incapable of growing on glycolate as the sole carbon source. When the overexpression of an enzyme in these deficient mutants restores the growth of the bacteria on the medium containing glycolate as the sole carbon source, it means that this enzyme encodes a functional equivalent to the E. coli glycolate dehydrogenase. The method and means for the complementation analysis is described in Bari et al, 2004, and incorporated herein by reference.

Polypeptides having the enzymatic activity of a glycolate dehydrogenase, and nucleic acids encoding them, have been identified from various sources, including bacteria, algae, and plants.

TABLE 1
Examples of known glycolate dehydrogenase enzymes.

| Enzyme | Characteristics |
|---|---|
| Glycolate dehydrogenase | Escherichia coli |
| (gi/1141710/gb/L43490.1/ECOGLCC) | Organic co-factors dependent |
| | Encoded by the glc operon |
| Glycolate dehydrogenase | Activity described for algal |
| (GDH; EC 1.1.99.14) | mitochondria |
| | Organic co-factors dependent |
| Glycolate dehydrogenase | Synechocystis cells |
| (s110404) | Organic co-factors dependent |
| Glycolate dehydrogenase | Arabidopsis thaliana |
| (At5g06580) | Targeted to mitochondria |
| | Organic co-factors dependent |

Nucleic acid molecules encoding one or more polypeptides having the enzymatic activity of a glycolate dehydrogenase may be isolated e.g. from genomic DNA or cDNA libraries produced from any origin, including bacterial, mammalian, algal, fungal, and plant origin. Alternatively, they may be produced by means of recombinant DNA techniques (e.g. PCR), or by means of chemical synthesis. The identification and isolation of such nucleic acid molecules may take place by using the sequences, or part of those sequences, of the known glycolate dehydrogenases nucleic acid molecules or, as the case may be, the reverse complement strands of these molecules, e.g. by hybridization according to standard methods (see e.g. Sambrook et al., 1989).

The glycolate dehydrogenase for the purpose of the invention can be any naturally-occurring glycolate dehydrogenase, or any active fragment thereof or any variant thereof wherein some amino acids (preferably 1 to 20 amino acids, more preferably 1 to 10, even more preferably 1 to 5) have been replaced, added or deleted such that the enzyme retains its glycolate dehydrogenase activity.

According to the invention, the glycolate dehydrogenase may be a chimeric glycolate dehydrogenase. The term “chimeric glycolate dehydrogenase” is intended to mean a glycolate dehydrogenase which is obtained by combining portions of enzymes from various origins, such as example the N-terminal portion of a first enzyme with the C-terminal portion of a second enzyme, so as to obtain a novel functional chimeric glycolate dehydrogenase, with each portion selected for its particular properties. As an example, a functional chimeric glycolate dehydrogenase may be generated in order to combine an efficient active site coming from a first glycolate dehydrogenase with a good stability in rice provided by a second glycolate dehydrogenase.

According to the present invention, a “nucleic acid” or “nucleic acid molecule” is understood as being a polynucleotide molecule which can be of the DNA or RNA type, preferably of the DNA type, and in particular double-stranded. It can be of natural or synthetic origin. Synthetic nucleic acids are generated in vitro. Examples of such synthetic nucleic acids are those in which the codons which encode polypeptide(s) having the enzymatic activity of a glycolate dehydrogenase according to the invention have been optimized in accordance with the host organism in which it is to be expressed (e.g., by replacing codons with those codons more preferred or most preferred in codon usage tables of such host organism or the group to which such host organism belongs, compared to the original host). Methods for codon optimization are well known to the skilled person.

The glycolate dehydrogenase activity involved in the method of the invention may be obtained by one or more polypeptides. When said activity is obtained from more than one polypeptides, the nucleic acids encoding the polypeptides may be transferred to plant cells in a single plasmid construct or independently in several constructs.

Preferred polypeptides having the enzymatic activity of a glycolate dehydrogenase are those encoded by the E. coli glc operon (gi/1141710/gb/L43490.1/ECOGLCC). Most preferred are polypeptides which comprise the amino acid sequences of SEQ ID NOs: 2 (Glc D), 4 (Glc E) and 6 (Glc F). Accordingly, nucleic acids comprising a polynucleotide sequence of SEQ ID NOs: 1, 3 and 5 can be used for performing the present invention.

Alternatively, polypeptide(s) having the enzymatic activity of a glycolate dehydrogenase and derived from Arabidopsis thaliana or other higher plant sources may be used. A preferred Arabidopsis thaliana polypeptide comprises the amino acid sequence of SEQ ID NO: 8 and is encoded by a nucleic acid comprising the polynucleotide sequence of SEQ ID NO: 7. Accordingly, nucleic acids comprising a polynucleotide sequence of SEQ ID NO: 7 can be used for performing the present invention.

Alternatively, polypeptide(s) having the enzymatic activity of a glycolate dehydrogenase and derived from alga, and particularly from Chlamydomonas or from Synechocystis (Eisenhut et al., 2006) may be used. A preferred Chlamydomonas polypeptide comprises the amino acid sequence of SEQ ID NO 12 and is encoded by a nucleic acid comprising the polynucleotide sequence of SEQ ID NO 11. Accordingly, nucleic acids comprising a polynucleotide sequence of SEQ ID NO 11 can be used for performing the present invention. A preferred Synechocystis polypeptide comprises the amino acid sequence of SEQ ID NO 16 and is encoded by a nucleic acid comprising the polynucleotide sequence of SEQ ID NO 15. Accordingly, nucleic acids comprising a polynucleotide sequence of SEQ ID NO 15 can be used for performing the present invention.

In another embodiment of the invention, a truncated polypeptide which retained its glycolate dehydrogenase activity may be used. A preferred truncated Chlamydomonas polypeptide comprises the amino acid sequence of SEQ ID NO 14 and is encoded by a nucleic acid comprising the polynucleotide sequence of SEQ ID NO 13. Accordingly, nucleic acids comprising a polynucleotide sequence of SEQ ID NO 13 can be used for performing the present invention.

Since some changes to the amino acid sequences are possible without substantially changing the enzymatic activity of a glycolate dehydrogenase, any protein comprising an amino acid sequences substantially similar to SEQ ID NO: 2, 4, and 6, or SEQ ID NO: 8, or SEQ ID NO: 10, or SEQ ID NO: 12, or SEQ ID NO: 14, or SEQ ID NO: 16 wherein less than 20, preferably less than 10, more preferably 1 to 5, amino acids are replaced by other amino acids without substantially changing the glycolate dehydrogenase enzymatic activity, may be used in the method of the invention.

The method of the invention encompasses the introduction into the genome of a rice plant cell of one or more nucleic acids encoding one or more polypeptides having the enzymatic activity of a glycolate dehydrogenase, wherein said polypeptide(s) comprise(s) a sequence having a sequence identity of at least 60, 70, 80 or 90%, particularly at least 95%, 97%, 98% or at least 99% at the amino acid sequence level with SEQ ID NO: 2, 4, and 6, or with SEQ ID NO: 8, or with SEQ ID NO: 10, or with SEQ ID NO: 12, or SEQ ID NO: 14, or SEQ ID NO: 16, wherein the introduction of the nucleic acid(s) result in a de novo expression of at least one polypeptide having the enzymatic activity of a glycolate dehydrogenase, and wherein said activity is located inside the chloroplasts.

The method of the invention encompasses also the introduction into the genome of a rice plant cells of one or more nucleic acids encoding one or more polypeptides having the enzymatic activity of a glycolate dehydrogenase, wherein said one or more nucleic acids comprise nucleic acid sequence(s) with at least 60, 70, 80 or 90%, particularly at least 95%, 97%, 98% or at least 99%, sequence identity to the nucleotide sequence of SEQ ID NO: 1, 3, and 5, or SEQ ID NO: 7, or SEQ ID NO: 9, or SEQ ID NO: 11, or SEQ ID NO: 13, or SEQ ID NO: 15, wherein the introduction of the nucleic acid(s) result in a de novo expression of at least one polypeptide having the enzymatic activity of a glycolate dehydrogenase, and wherein said activity is located inside the chloroplasts.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences can be performed by the Needleman and Wunsch algorithm (Needleman and Wunsch 1970) in EMBOSS (Rice et al., 2000) to find optimum alignment over the entire length of the sequences, using default settings (gap opening penalty 10, gap extension penalty 0.5).

Once the sequence of a foreign DNA is known, primers and probes can be developed which specifically recognize these sequences in the nucleic acid (DNA or RNA) of a sample by way of a molecular biological technique. For instance, a PCR method can be developed to identify the genes used in the method of the invention (gdh genes) in biological samples (such as samples of plants, plant material or products comprising plant material). Such a PCR is based on at least two specific “primers”, e.g., both recognizing a sequence within the gdh coding region used in the invention (such as the coding region of SEQ ID No. 1, 3, 5, 7, 9, 11, 13 or 15), or one recognizing a sequence within the gdh coding region and the other recognizing a sequence within the associated transit peptide sequence or within the regulatory regions such as the promoter or 3′ end of the chimeric gene comprising a gdh DNA used in the invention. The primers preferably have a sequence of between 15 and 35 nucleotides which under optimized PCR conditions specifically recognize a sequence within the gdh chimeric gene used in the invention, so that a specific fragment (“integration fragment” or discriminating amplicon) is amplified from a nucleic acid sample comprising a gdh gene used in the invention. This means that only the targeted integration fragment, and no other sequence in the plant genome or foreign DNA, is amplified under optimized PCR conditions.

The method of the invention encompasses also the introduction into the genome of a rice plant cell of one or more nucleic acids encoding one or more polypeptides having the enzymatic activity of a glycolate dehydrogenase, wherein said one or more nucleic acids comprise one or more nucleic acids hybridizing under stringent conditions to a nucleotide sequence selected from the group of SEQ ID NO 1, 3, and 5, SEQ ID NO 7, SEQ ID NO 9, SEQ ID NO 11, SEQ ID NO 13, and SEQ ID NO 15 wherein the introduction of the nucleic acid(s) result in a de novo expression of at least one polypeptide having the enzymatic activity of a glycolate dehydrogenase, and wherein said activity is located inside the chloroplasts. Stringent hybridization conditions, as used herein, refers particularly to the following conditions: immobilizing the relevant DNA sequences on a filter, and prehybridizing the filters for either 1 to 2 hours in 50% formamide, 5% SSPE, 2×Denhardt's reagent and 0.1% SDS at 42° C., or 1 to 2 hours in 6×SSC, 2×Denhardt's reagent and 0.1% SDS at 68° C. The denatured dig- or radio-labeled probe is then added directly to the prehybridization fluid and incubation is carried out for 16 to 24 hours at the appropriate temperature mentioned above. After incubation, the filters are then washed for 30 minutes at room temperature in 2×SSC, 0.1% SDS, followed by 2 washes of 30 minutes each at 68° C. in 0.5×SSC and 0.1% SDS. An autoradiograph is established by exposing the filters for 24 to 48 hours to X-ray film (Kodak MR-2 or equivalent) at −70° C. with an intensifying screen. Of course, equivalent conditions and parameters can be used in this process while still retaining the desired stringent hybridization conditions.

The terminology DNA or protein “comprising” a certain sequence X, as used throughout the text, refers to a DNA or protein including or containing at least the sequence X, so that other nucleotide or amino acid sequences can be included at the 5′ (or N-terminal) and/or 3′ (or C-terminal) end, e.g. (the nucleotide sequence encoding) a selectable marker protein, (the nucleotide sequence encoding) a transit peptide, and/or a 5′ leader sequence or a 3′ trailer sequence. Similarly, use of the term “comprise”, “comprising” or “comprises” throughout the text and the claims of this application should be understood to imply the inclusion of a stated integer or step or group of integers or steps but not the exclusion of any other integer or step or group of integers or steps

The method of the present invention consists in installing a glycolate dehydrogenase activity inside the chloroplast. This can be done either by introducing the nucleic acid(s) encoding the glycolate dehydrogenase activity into the nuclear genome of plant cells, the coding sequence(s) of the protein then being fused to a nucleic acid encoding a chloroplast transit peptide. Alternatively, the glycolate dehydrogenase activity can be put into the chloroplast by direct transformation of the chloroplast genome with the nucleic acid(s) encoding the corresponding enzyme.

General techniques for transforming plant cells or plants tissues, in particular rice plant cells are well known in the art. One series of methods comprises bombarding cells, protoplasts or tissues with particles to which the DNA sequences are attached. Another series of methods comprises using, as the means for transfer into the plant, a chimeric gene which is inserted into an Agrobacterium tumefaciens Ti plasmid or an Agrobacterium rhizogenes Ri plasmid. Other methods may be used such as microinjection or electroporation or otherwise direct precipitation using PEG. The skilled person can select any appropriate method and means for transforming the plant cell or the plant, in particular rice plant cells or plants. For rice, agrobacterium-mediated transformation (Hiei et al., 1994, and Hiei et al., 1997, incorporated herein by reference), electroporation (U.S. Pat. No. 5,641,664 and U.S. Pat. No. 5,679,558, incorporated herein by reference), or bombardment (Christou et al., 1991, incorporated herein by reference) could be advantageously performed. A suitable technology for transformation of monocotyledonous plants, and particularly rice, is described in WO 92/09696, incorporated herein by reference.

For the purpose of expressing the nucleic acid(s) which encode the polypeptide(s) having the enzymatic activity as required for the present invention in plant cells, any convenient regulatory sequences can be used. The regulatory sequences will provide transcriptional and translational initiation as well as termination regions, where the transcriptional initiation may be constitutive or inducible. The coding region is operably linked to such regulatory sequences. Suitable regulatory sequences are represented by the constitutive 35S promoter. Alternatively, the constitutive ubiquitin promoter can be used, in particular the maize ubiquitin promoter (GenBank: gi19700915). Examples for inducible promoters represent the light inducible promoters of the small subunit of RUBISCO and the promoters of the “light harvesting complex binding protein (Ihcb)”. Advantageously, the promoter region of the gos2 gene of Oryza sativa including the 5′ UTR of the GOS2 gene with intron (de Pater et al., 1992), the promoter region of the ribulose-1,5-biphosphate carboxylase small subunit gene of Oryza sativa (Kyozuka J. et al., 1993), or the promoter region of the actin 1 gene of Oryza sativa (McElroy D. et al., 1990) may be used.

According to the invention, use may also be made, in combination with the promoter, of other regulatory sequences, which are located between the promoter and the coding sequence, such as transcription activators (“enhancers”), for instance the translation activator of the tobacco mosaic virus (TMV) described in Application WO 87/07644, or of the tobacco etch virus (TEV) described by Carrington & Freed 1990, for example, or introns such as the adh1 intron of maize or intron 1 of rice actin.

As a regulatory terminator or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in Application EP 0 633 317.

In one particular embodiment of the invention whereby transformation of the nuclear genome is preferred, a nucleic acid which encodes a chloroplast transit peptide is employed 5′ of the nucleic acid sequence encoding a glycolate dehydrogenase, with this transit peptide sequence being arranged between the promoter region and the nucleic acid encoding the glycolate dehydrogenase so as to permit expression of a transit peptide/glycolate dehydrogenase fusion protein. The transit peptide makes it possible to direct the glycolate dehydrogenase into the plastids, more especially the chloroplasts, with the fusion protein being cleaved between the transit peptide and the glycolate dehydrogenase when the latter enters the plastid. The transit peptide may be a single peptide, such as an EPSPS transit peptide (described in U.S. Pat. No. 5,188,642) or a transit peptide of the plant ribulose biscarboxylase/oxygenase small subunit (RuBisCO ssu), for example the chloroplast transit peptide derived from the ribulose-1,5-bisphosphate carboxylase gene from Solanum tuberosum (GenBank: G68077, amino acids 1-58), where appropriate including a few amino acids of the N-terminal part of the mature RuBisCO ssu (EP 189 707), or the chloroplast targeting peptide of the potato rbcS1 gene (gi21562). A transit peptide may be the whole naturally occurring (wild-type) transit peptide, a functional fragment thereof, a functional mutant thereof. It can also be a chimeric transit peptide wherein at least two transit peptides are associated to each other or wherein parts of different transit peptides are associated to each other in a functional manner. One example of such chimeric transit peptide comprises a transit peptide of the sunflower RuBisCO ssu fused to the N-terminal part of the maize RuBisCO ssu, fused to the transit peptide of the maize RuBisCO ssu, as described in patent EP 508 909.

The person skilled in the art will be able to construct nucleic acid suitable for performing the invention comprising a nucleic acid encoding a mature (i.e. without transit peptide) glycolate hydroxylase, optimized or not for the expression in rice and wherein the first ATG codon, if any, may or may not be deleted, operably-linked to a chloroplast transit peptide. An example of such nucleic acid suitable for performing the invention may be the Arabidopsis thaliana glycolate dehydrogenase DNA sequence optimized for the expression in rice operably-linked to the sequence encoding a chimeric chloroplast transit peptide, as described in SEQ ID NO 9.

Alternatively, the polypeptides may be directly expressed into the chloroplast using transformation of the chloroplast genome. Methods for integrating nucleic acids of interest into the chloroplast genome are known in the art, in particular methods based on the mechanism of homologous recombination. Suitable vectors and selection systems are known to the person skilled in the art. The coding sequences for the polypeptides may either be transferred in individual vectors or in one construct, where the individual open reading frames may be fused to one or several polycistronic RNAs with ribosome binding sites added in front of each individual open reading frame in order to allow independent translation. An example of means and methods which can be used for such integration into the chloroplast genome is given for example in WO 06/108830, the content of which are hereby incorporated by reference. When the nucleic acids are directly integrated into the chloroplast genome, a transit peptide sequence is not required. In that case, the (Met) translation start codon may be added to the sequence encoding a mature protein to ensure initiation of translation.

Subject-matter of the present invention also are rice plant cells, rice plant tissues or rice plants comprising one or more nucleic acids expressing inside the chloroplast one or more polypeptides having the enzymatic activity of glycolate dehydrogenase.

Preferred embodiments of the nucleic acids introduced into the rice plant cells, rice plant tissues or rice plants are mentioned above.

Rice plant cell is understood, according to the invention, as being any cell which is derived from or found in a Oriza sativa plant and which is able to form or is part of undifferentiated tissues, such as calli, differentiated tissues such as embryos, parts of plants, plants or seeds.

The present invention also relates to rice plants which contain transformed cells, in particular plants which are regenerated from the transformed cells. The regeneration can be obtained by any appropriate method. The following patents and patent applications may be cited, in particular, with regard to the methods for transforming plant cells and regenerating plants: U.S. Pat. No. 4,459,355, U.S. Pat. No. 4,536,475, U.S. Pat. No. 5,464,763, U.S. Pat. No. 5,177,010, U.S. Pat. No. 5,187,073, EP 267,159, EP 604 662, EP 672 752, U.S. Pat. No. 4,945,050, U.S. Pat. No. 5,036,006, U.S. Pat. No. 5,100,792, U.S. Pat. No. 5,371,014, U.S. Pat. No. 5,478,744, U.S. Pat. No. 5,179,022, U.S. Pat. No. 5,565,346, U.S. Pat. No. 5,484,956, U.S. Pat. No. 5,508,468, U.S. Pat. No. 5,538,877, U.S. Pat. No. 5,554,798, U.S. Pat. No. 5,489,520, U.S. Pat. No. 5,510,318, U.S. Pat. No. 5,204,253, U.S. Pat. No. 5,405,765, EP 442 174, EP 486 233, EP 486 234, EP 539 563, EP 674 725, WO 91/02071 and WO 95/06128.

The present invention also relates to transformed plants or part thereof, which are derived by cultivating and/or crossing the above regenerated plants, and to the seeds of the transformed plants, characterized in that they contain a transformed plant cell according to the invention. The present invention also relates to any products such as the meal which are obtained by processing the plants, part thereof, or seeds of the invention. For example, the invention encompasses rice grains obtained from the processing of the rice seeds according to the invention, but also meal obtained from the further processing of the rice seeds or the rice grains, as well as any food product obtained from said meal.

### SEQUENCE LISTING

SEQ ID NO 1: Escherichia coli gcl D DNA sequence

SEQ ID NO 2: amino acid sequence encoded by SEQ ID NO 1

SEQ ID NO 3: Escherichia coli gcl E DNA sequence

SEQ ID NO 4: amino acid sequence encoded by SEQ ID NO 3

SEQ ID NO 5: Escherichia coli gcl F DNA sequence

SEQ ID NO 6: amino acid sequence encoded by SEQ ID NO 5

SEQ ID NO 7: DNA sequence encoding the mature (i.e. without transit peptide) Arabidopsis thaliana glycolate dehydrogenase, optimized for the expression in rice.

SEQ ID NO 8: amino acid sequence encoded by SEQ ID NO 7

SEQ ID NO 9: optimized Arabidopsis thaliana glycolate dehydrogenase DNA sequence operably-linked to the sequence encoding an optimized chloroplast transit peptide.

SEQ ID NO 10: amino acid sequence encoded by SEQ ID NO 9.

SEQ ID NO11: DNA sequence encoding the mature (i.e. without transit peptide) Chlamydomonas glycolate dehydrogenase

SEQ ID NO 12: amino acid sequence encoded by SEQ ID NO 11

SEQ ID NO 13: DNA sequence encoding a truncated Chlamydomonas glycolate dehydrogenase

SEQ ID NO 14: amino acid sequence encoded by SEQ ID NO 13

SEQ ID NO15: DNA sequence encoding Synechocystis glycolate dehydrogenase

SEQ ID NO 16: amino acid sequence encoded by SEQ ID NO 15

### EXAMPLES

### Ex 1

### Construction of Plant Expression Vectors Encoding E. coli GDH

The coding sequences for the glcD, g/cE and glcF (gi/1141710/gb/L43490.1/ECOGLCC) subunits of glycolate dehydrogenase from Escherichia coli were obtained by chemical DNA synthesis. Plasmid pTTS84 contained three expression cassettes, encoding the three E. coli GDH subunits: glcE was driven by the promoter region of the gos2 gene of Oryza sativa (rice) as described by de Pater et al. (1992), including the 5′ UTR of the GOS2 gene with intron; glcF was driven by the promoter region of the ribulose-1,5-biphosphate carboxylase small subunit gene of Oryza sativa (rice) as described by Kyozuka et al. (1993); glcD was driven by the promoter region of the actin 1 gene of Oryza sativa (rice) (Mc Elroy et al., 1990). Each of the three E. coli GDH subunit genes contained a sequence encoding the optimized transit peptide (OTP) chloroplast targeting sequence as described in EP 0508909. Plasmid pTTS84 also contained a bar expression cassette including a p35S promoter and a 3′nos terminator region.

### Ex 2

### Construction of Plant Expression Vectors Encoding Arabidopsis GDH

The coding sequence for the GDH coding region from Arabidopsis thaliana (At5g06580) was obtained by chemical DNA synthesis. In the design of the synthetic gene, the sequence encoding the putative mitochondrial targeting sequence was excluded, and replaced by the sequence encoding the OTP chloroplast targeting sequence. Different vectors were made using this synthetic gene. In plasmid pTTS86, the gene was driven by the p35S promoter, while in plasmid pTTS87, the promoter region of the ribulose-1,5-biphosphate carboxylase small subunit gene of Oryza sativa (rice) as described by Kyozuka et al. (1993) was used. Both plasmids also contained a bar expression cassette including a p35S promoter and a 3′nos terminator region.

### Ex 3

### Plant Transformation and Regeneration

The acceptor Agrobacterium strain ACH5C3(pGV4000) carried a non-oncogenic (disarmed) Ti plasmid from which the T-region has been deleted. This Ti plasmid carried the necessary vir gene functions that are required for transfer of the T-DNA region of the intermediate cloning vector to the plant genome.

The intermediate cloning vector (e.g. pTTS84, pTTS86, pTTS87) was constructed in Escherichia coli. It was transferred to the acceptor Agrobacterium tumefaciens strain via a heat shock. Agrobacterium-mediated gene transfer of the intermediate cloning vector(s) resulted in transfer of the DNA fragment between the T-DNA border repeats to the plant genome.

As target tissue for transformation, immature embryo or embryo-derived callus derived from japonica and indica rice cultivars which has been cut into small pieces, essentially using the technique described in PCT patent publication WO 92/09696. Agrobacterium was co-cultivated with the rice tissues for some days, and then removed by suitable antibiotics. Transformed rice cells were selected by addition of glufosinate ammonium (with phosphinothricin 5 mg/L) to the rice tissue culture medium.

Calli growing on media with glufosinate ammonium were transferred to regeneration medium. When plantlets with roots and shoots had developed, they were transferred to soil, and placed in the greenhouse.

### Ex 4

### Chloroplast Isolation and Enzymatic Assays

Intact chloroplasts are isolated using the procedure described by Kleffmann et al., 2007. These preparations are free of contaminating catalase and fumarase activity (>95% purity).

Glycolate dehydrogenase activities are measured as described in Lord J. M. 1972. 100 μg of chloroplast protein extract is added to 100 μmol potassium phosphate (pH 8.0), 0.2 μmol DCIP, 0.1 ml 1% (w/v) PMS, and 10 μmol potassium glycolate in a final volume of 2.4 ml. At fixed time intervals, individual assays are terminated by the addition of 0.1 ml of 12 M HCl. After standing for 10 min, 0.5 ml of 0.1 M phenylhydrazine-HCl is added. The mixture is allowed to stand for a further 10 min, and then the extinction due to the formation of glyoxylate phenylhydrazone is measured at 324 nm.

### Ex 5

### CO2 Release from Labeled Glycolate in Chloroplasts Extracts

1 μCi of [1,2-14C]-glycolate (Hartmann Analytics) is added to 50 μg of chloroplast protein extract in a tightly closed 15-ml reaction tube. Released CO2 is absorbed in a 500-μl reaction tube containing 0.5 M NaOH attached to the inner wall of the 15-ml tube. Samples are incubated for 5 h and the gas phase in the reaction tube is frequently mixed with a syringe.

### CITED REFERENCES

Bari et al., 2004, J. of Experimental Botany, Vol 55, No 397, 623-630
Deblaere et al., 1985, Nucl. Acids Res. 13, 4777-4788
De Pater et al., 1992, The Plant J. 2: 837
Eisenhut et al., 2006, Plant Phys., 142:333-342
Gehlen J. et al, 1996, Plant Mol Biol. 32:831-48
Kebeish R. et al., 2007, Nature Biotechnology, vol 25, No 5, 593-599
Kleffmann et al., 2007, Plant Physiology, 143, 912-923
Ku et al., 1999, Nat Biotechnol 17: 76-80
Kyozuka J. et al., 1993, Plant Physiology 102: 991-1000
Leegood R. C. et al, 1995, J. exp. Bot. 46, 1397-1414
Lord J. M. 1972, Biochim. Biophys. Acta 267, 227-237
McElroy D. et al., 1990, The Plant Cell 2: 163-171
Matsuoka et al., 2001, Molecular engineering of C₄ photosynthesis. Annu Rev Plant Physio. Plant Mol Biol 52: 297-314
Needleman et al., 1970, J. Mol. Biol. 48:443-53
Rice et al., 2000, Trends in Genetics, 16:276-277
Sambrook et al., 1989, Molecular Cloning, A Laboratory Manual, 2nd Edition, Cold Spring Harbor Laboratory Press, Cold Spring Harbor, N.Y
Sharkey T. D., 1988, Physiol. Plant., 73, 147-152
Suzuki et al., 2000, Plant Physiol., 124:163-72
Taniguchi Y. et al., 2008, Journal of Experimental Botany, In press.
Zhu X. G. et al., 2008, Current opinion in Biotechnology, 19:153-159

1. A method for increasing biomass production and/or seed production and/or carbon fixation in rice plants comprising introducing into the genome of a rice plant cell a nucleic acid sequence encoding a polypeptide having the enzymatic activity of a plant glycolate dehydrogenase, wherein the introduction of said nucleic acid sequence results in a de novo expression of a polypeptide having the enzymatic activity of a glycolate dehydrogenase and wherein said polypeptide is localized in chloroplasts of the produced plant.

2. The method of claim 1, wherein said introduction of said nucleic acid sequence is done into the nuclear genome of the rice plant cells, and wherein said nucleic acid sequence encodes a polypeptide comprising an amino acid fragment that targets the polypeptide to the chloroplast.

3. The method of claim 1, wherein said polypeptide having the enzymatic activity of a plant glycolate dehydrogenase comprises an Arabidopsis glycolate dehydrogenase.

4. The method of claim 1, wherein said polypeptide comprises the sequence of SEQ ID NO: 8.

5. The method of claim 1, wherein said nucleic acid sequence comprises a polynucleotide sequence having at least 97% sequence identity to the polynucleotide sequence of SEQ ID NO: 7.

6. The method of claim 5, wherein said polynucleotide comprises SEQ ID NO: 7.

7. A transgenic rice plant transformed with a polynucleotide sequence, said polynucleotide sequence comprising a nucleic acid sequence encoding a polypeptide having the enzymatic activity of a plant glycolate dehydrogenase, operably linked with regulatory sequences for expressing said nucleic acid sequence encoding a polypeptide, wherein said polypeptide is localized in the chloroplasts of said rice plant.

8. The rice plant of claim 7 wherein said polypeptide further comprises an amino acid sequence which targets said polypeptide to the chloroplast.

9. The rice plant of claim 7 wherein said polypeptide having the enzymatic activity of a plant glycolate dehydrogenase comprises an Arabidopsis glycolate dehydrogenase.

10. The rice plant of claim 7 wherein said polypeptide comprises the amino acid sequence of SEQ ID NO: 8.

11. The rice plant of claim 7 wherein said nucleic acid sequence comprises a polynucleotide sequence having at least 97% sequence identity to the polynucleotide sequence of SEQ ID NO: 7.

12. The rice plant of claim 11 wherein said polynucleotide comprises SEQ ID NO: 7.

13. Rice seed, characterized in that it has been obtained from a transformed plant according to claim 7 and contains said polynucleotide sequence.

14. Rice grain obtained from the processing of the rice seed of claim 13.

15. Meal derived from the processing of the rice seed of claim 13 or from rice grain obtained from the processing of the rice seed of claim 13.

16. Food product obtained from the meal of claim 15.


SEQUENCE LISTING (16 sequences)

SEQ ID NO: 1 - DNA, 1500 bp, Escherichia coli
  CDS (1)..(1497): gcl D
atgagcatcttgtacgaagagcgtcttgatggcgctttacccgatgtcgaccgcacatcggtactgatggcactgcgtgagcatgtccctggacttgagatcctgcataccgatgaggagatcattccttacgagtgtgacgggttgagcgcgtatcgcacgcgtccattactggttgttctgcctaagcaaatggaacaggtgacagcgattctggctgtctgccatcgcctgcgtgtaccggtggtgacccgtggtgcaggcaccgggctttctggtggcgcgctgccgctggaaaaaggtgtgttgttggtgatggcgcgctttaaagagatcctcgacattaaccccgttggtcgccgcgcgcgcgtgcagccaggcgtgcgtaacctggcgatctcccaggccgttgcaccgcataatctctactacgcaccggacccttcctcacaaatcgcctgttccattggcggcaatgtggctgaaaatgccggcggcgtccactgcctgaaatatggtctgaccgtacataacctgctgaaaattgaagtgcaaacgctggacggcgaggcactgacacttggatcggacgcgctggattcacctggttttgacctgctggcgctgttcaccggatcggaaggtatgctcggcgtgaccaccgaagtgacggtaaaactgctgccgaagccgcccgtggcgcgggttctgttagccagctttgactcggtagaaaaagccggacttgcggttggtgacatcatcgccaatggcattatccccggcgggctggagatgatggataacctgtcgatccgcgcggcggaagattttattcatgccggttatcccgtcgacgccgaagcgattttgttatgcgagctggacggcgtggagtctgacgtacaggaagactgcgagcgggttaacgacatcttgttgaaagcgggcgcgactgacgtccgtctggcacaggacgaagcagagcgcgtacgtttctgggccggtcgcaaaaatgcgttcccggcggtaggacgtatctccccggattactactgcatggatggcaccatcccgcgtcgcgccctgcctggcgtactggaaggcattgcccgtttatcgcagcaatatgatttacgtgttgccaacgtctttcatgccggagatggcaacatgcacccgttaatccttttcgatgccaacgaacccggtgaatttgcccgcgcggaagagctgggcgggaagatcctcgaactctgcgttgaagttggcggcagcatcagtggcgaacatggcatcgggcgagaaaaaatcaatcaaatgtgcgcccagttcaacagcgatgaaatcacgaccttccatgcggtcaaggcggcgtttgaccccgatggtttgctgaaccctgggaaaaacattcccacgctacaccgctgtgctgaatttggtgccatgcatgtgcatcacggtcatttacctttccctgaactggagcgtttctga

SEQ ID NO: 2 - protein, 499 aa, Escherichia coli
MSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 3 - DNA, 1053 bp, Escherichia coli
  CDS (1)..(1050): gcl E
atgctacgcgagtgtgattacagccaggcgctgctggagcaggtgaatcaggcgattagcgataaaacgccgctggtgattcagggcagcaatagcaaagcctttttaggtcgccctgtcaccgggcaaacgctggatgttcgttgtcatcgcggcattgttaattacgacccgaccgagctggtgataaccgcgcgtgtcggaacgccgctggtgacaattgaagcggcgctggaaagcgcggggcaaatgctcccctgtgagccgccgcattatggtgaagaagccacctggggcgggatggtcgcctgcgggctggcggggccgcgtcgcccgtggagcggttcggtccgcgattttgtcctcggcacgcgcatcattaccggcgctggaaaacatctgcgttttggtggcgaagtgatgaaaaacgttgccggatacgatctctcacggttaatggtcggaagctacggttgtcttggcgtgctcactgaaatctcaatgaaagtgttaccgcgaccgcgcgcctccctgagcctgcgtcgggaaatcagcctgcaagaagccatgagtgaaatcgccgagtggcaactccagccattacccattagtggcttatgttacttcgacaatgcgttgtggatccgccttgagggcggcgaaggatcggtaaaagcagcgcgtgaactgctgggtggcgaagaggttgccggtcagttctggcagcaattgcgtgaacaacaactgccgttcttctcgttaccaggtaccttatggcgcatttcattacccagtgatgcgccgatgatggatttacccggcgagcaactgatcgactggggcggggcgttacgctggctgaaatcgacagccgaggacaatcaaatccatcgcatcgcccgcaacgctggcggtcatgcgacccgctttagtgccggagatggtggctttgccccgctatcggctcctttattccgctatcaccagcagcttaaacagcagctcgacccttgcggcgtgtttaaccccggtcgcatgtacgcggaactttga

SEQ ID NO: 4 - protein, 350 aa, Escherichia coli
MLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 5 - DNA, 1224 bp, Escherichia coli
  CDS (1)..(1221): gcl F
atgcaaacccaattaactgaagagatgcggcagaacgcgcgcgcgctggaagccgacagcatcctgcgcgcctgtgttcactgcggattttgtaccgcaacctgcccaacctatcagcttctgggcgatgaactggacgggccgcgcgggcgcatctatctgattaaacaggtgctggaaggcaacgaagtcacgcttaaaacacaggagcatctcgatcgctgcctcacttgccgtaattgtgaaaccacctgtccttctggtgtgcgctatcacaatttgctggatatcgggcgtgatattgtcgagcagaaagtgaaacgcccactgccggagcgaatactgcgcgaaggattgcgccaggtagtgccgcgtccggcggtcttccgtgcgctgacgcaggtagggctggtgctgcgaccgtttttaccggaacaggtcagagcaaaactgcctgctgaaacggtgaaagctaaaccgcgtccgccgctgcgccataagcgtcgggttttaatgttggaaggctgcgcccagcctacgctttcgcccaacaccaacgcggcaactgcgcgagtgctggatcgtctggggatcagcgtcatgccagctaacgaagcaggctgttgtggcgcggtggactatcatcttaatgcgcaggagaaagggctggcacgggcgcgcaataatattgatgcctggtggcccgcgattgaagcaggtgccgaggcaattttgcaaaccgccagcggctgcggcgcgtttgtcaaagagtatgggcagatgctgaaaaacgatgcgttatatgccgataaagcacgtcaggtcagtgaactggcggtcgatttagtcgaacttctgcgcgaggaaccgctggaaaaactggcaattcgcggcgataaaaagctggccttccactgtccgtgtaccctacaacatgcgcaaaagctgaacggcgaagtggaaaaagtgttgcttcgtcttggatttaccttaacggacgttcccgacagccatctgtgctgcggttcagcgggaacatatgcgttaacgcatcccgatctggcacgccagctgcgggataacaaaatgaatgcgctggaaagcggcaaaccggaaatgatcgtcaccgccaacattggttgccagacgcatctggcgagcgccggtcgtacctctgtgcgtcactggattgaaattgtagaacaagcccttgaaaaggaataa

SEQ ID NO: 6 - protein, 407 aa, Escherichia coli
MQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 7 - DNA, 1614 bp, artificial
  DNA sequence encoding mature (without transit peptide) Arabidopsis thaliana glycolate dehydrogenase, optimized for expression in rice
ggtgatgttacagtgctctcgccagttaagggcaggagaaggctccctacctgttggagcagcagtctctttccgctggcgatagcagccagcgcaactagcttcgcctacctgaacctctcgaacccgagcatcagcgaaagttcctcggccctagactccagggacatcactgtgggcgggaaagatagcacggaggccgtggtgaaaggcgagtacaagcaggtgccgaaggagctcatcagccagctgaagaccatcctggaggacaacctcacgaccgactacgacgaacgctacttccacgggaagccgcagaacagcttccacaaggccgtgaacattccggacgtcgtggtgttccccagaagcgaggaggaggtgagcaagatcctcaagagctgcaacgagtacaaggtgcccatcgtgccatatggaggtgccacaagcatcgagggccacacactagccccaaaaggaggcgtgtgcatcgacatgagcctcatgaaacgtgtgaaggcgctccacgtcgaggacatggacgtcatcgtggaaccgggaatcggctggctggaactcaacgagtacctggaggagtacgggctcttctttcccctcgatcctggtccaggcgcatcgatcgggggcatgtgtgcaactaggtgcagcggatccctcgcagttaggtacggcaccatgagggacaatgtgatcagcctcaaggtggtcctccccaatggagacgtggtcaagactgcgagcagggctaggaaatctgccgctggctacgacctgacgaggctcatcataggctccgagggaacactcggcgtgatcacggagatcaccctgaggctccaaaagatcccgcagcactcagtcgtggcggtctgcaacttcccgaccgttaaggatgccgcggatgtggccatcgcaaccatgatgagcggcatccaggtgagccgagttgagcttctcgacgaggtccagatccgagcgatcaacatggccaacgggaaaaacctgacggaggccccaacgctcatgttcgagttcatcggcacggaggcgtacaccagggaacagacccagatcgtccagcagatcgcgagcaagcacaacggcagcgacttcatgttcgcggaagagcctgaggcgaagaaggagctctggaagatcaggaaggaggcgctctgggcatgctatgcaatggcaccgggccatgaggcgatgattacggacgtctgtgtcccactcagccatctcgccgagctcatatccaggagcaagaaggagctcgacgcctcttccctgctctgcacagtgatcgcacatgccggagacgggaactttcacacgtgcatcatgttcgacccgagctcggaagagcaacgaagggaagcggagaggctgaaccacttcatggtccacagcgcgctcagcatggatgggacttgtactggcgaacatggcgtggggaccggcaagatgaagtacctggagaaggagctgggtatcgaggccctgcagaccatgaagcgcatcaagaagaccctcgacccgaacgacatcatgaacccagggaagctgatacccccgcatgtgtgcttctga

SEQ ID NO: 8 - protein, 537 aa, artificial
  Synthetic Construct
GDVTVLSPVKGRRRLPTCWSSSLFPLAIAASATSFAYLNLSNPSISESSSALDSRDITVGGKDSTEAVVKGEYKQVPKELISQLKTILEDNLTTDYDERYFHGKPQNSFHKAVNIPDVVVFPRSEEEVSKILKSCNEYKVPIVPYGGATSIEGHTLAPKGGVCIDMSLMKRVKALHVEDMDVIVEPGIGWLELNEYLEEYGLFFPLDPGPGASIGGMCATRCSGSLAVRYGTMRDNVISLKVVLPNGDVVKTASRARKSAAGYDLTRLIIGSEGTLGVITEITLRLQKIPQHSVVAVCNFPTVKDAADVAIATMMSGIQVSRVELLDEVQIRAINMANGKNLTEAPTLMFEFIGTEAYTREQTQIVQQIASKHNGSDFMFAEEPEAKKELWKIRKEALWACYAMAPGHEAMITDVCVPLSHLAELISRSKKELDASSLLCTVIAHAGDGNFHTCIMFDPSSEEQRREAERLNHFMVHSALSMDGTCTGEHGVGTGKMKYLEKELGIEALQTMKRIKKTLDPNDIMNPGKLIPPHVCF

SEQ ID NO: 9 - DNA, 1986 bp, Artificial
  DNA sequence encoding a Arabidopsis thaliana GDH optimized for expression in rice linked to an optimized chloroplast transit peptide
atggcttcgatctcctcctcagtcgcgaccgttagccggaccgcccctgctcaggccaacatggtggctccgttcaccggccttaagtccaacgccgccttccccaccaccaagaaggctaacgacttctccacccttcccagcaacggtggaagagttcaatgtatgcaggtgtggccggcctacggcaacaagaagttcgagacgctgtcgtacctgccgccgctgtctatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcggtgatgttacagtgctctcgccagttaagggcaggagaaggctccctacctgttggagcagcagtctctttccgctggcgatagcagccagcgcaactagcttcgcctacctgaacctctcgaacccgagcatcagcgaaagttcctcggccctagactccagggacatcactgtgggcgggaaagatagcacggaggccgtggtgaaaggcgagtacaagcaggtgccgaaggagctcatcagccagctgaagaccatcctggaggacaacctcacgaccgactacgacgaacgctacttccacgggaagccgcagaacagcttccacaaggccgtgaacattccggacgtcgtggtgttccccagaagcgaggaggaggtgagcaagatcctcaagagctgcaacgagtacaaggtgcccatcgtgccatatggaggtgccacaagcatcgagggccacacactagccccaaaaggaggcgtgtgcatcgacatgagcctcatgaaacgtgtgaaggcgctccacgtcgaggacatggacgtcatcgtggaaccgggaatcggctggctggaactcaacgagtacctggaggagtacgggctcttctttcccctcgatcctggtccaggcgcatcgatcgggggcatgtgtgcaactaggtgcagcggatccctcgcagttaggtacggcaccatgagggacaatgtgatcagcctcaaggtggtcctccccaatggagacgtggtcaagactgcgagcagggctaggaaatctgccgctggctacgacctgacgaggctcatcataggctccgagggaacactcggcgtgatcacggagatcaccctgaggctccaaaagatcccgcagcactcagtcgtggcggtctgcaacttcccgaccgttaaggatgccgcggatgtggccatcgcaaccatgatgagcggcatccaggtgagccgagttgagcttctcgacgaggtccagatccgagcgatcaacatggccaacgggaaaaacctgacggaggccccaacgctcatgttcgagttcatcggcacggaggcgtacaccagggaacagacccagatcgtccagcagatcgcgagcaagcacaacggcagcgacttcatgttcgcggaagagcctgaggcgaagaaggagctctggaagatcaggaaggaggcgctctgggcatgctatgcaatggcaccgggccatgaggcgatgattacggacgtctgtgtcccactcagccatctcgccgagctcatatccaggagcaagaaggagctcgacgcctcttccctgctctgcacagtgatcgcacatgccggagacgggaactttcacacgtgcatcatgttcgacccgagctcggaagagcaacgaagggaagcggagaggctgaaccacttcatggtccacagcgcgctcagcatggatgggacttgtactggcgaacatggcgtggggaccggcaagatgaagtacctggagaaggagctgggtatcgaggccctgcagaccatgaagcgcatcaagaagaccctcgacccgaacgacatcatgaacccagggaagctgatacccccgcatgtgtgcttctga

SEQ ID NO: 10 - protein, 661 aa, Artificial
  Synthetic Construct
MASISSSVATVSRTAPAQANMVAPFTGLKSNAAFPTTKKANDFSTLPSNGGRVQCMQVWPAYGNKKFETLSYLPPLSMAPTVMMASSATAVAPFQGLKSTASLPVARRSSRSLGNVSNGGRIRCGDVTVLSPVKGRRRLPTCWSSSLFPLAIAASATSFAYLNLSNPSISESSSALDSRDITVGGKDSTEAVVKGEYKQVPKELISQLKTILEDNLTTDYDERYFHGKPQNSFHKAVNIPDVVVFPRSEEEVSKILKSCNEYKVPIVPYGGATSIEGHTLAPKGGVCIDMSLMKRVKALHVEDMDVIVEPGIGWLELNEYLEEYGLFFPLDPGPGASIGGMCATRCSGSLAVRYGTMRDNVISLKVVLPNGDVVKTASRARKSAAGYDLTRLIIGSEGTLGVITEITLRLQKIPQHSVVAVCNFPTVKDAADVAIATMMSGIQVSRVELLDEVQIRAINMANGKNLTEAPTLMFEFIGTEAYTREQTQIVQQIASKHNGSDFMFAEEPEAKKELWKIRKEALWACYAMAPGHEAMITDVCVPLSHLAELISRSKKELDASSLLCTVIAHAGDGNFHTCIMFDPSSEEQRREAERLNHFMVHSALSMDGTCTGEHGVGTGKMKYLEKELGIEALQTMKRIKKTLDPNDIMNPGKLIPPHVCF

SEQ ID NO: 11 - DNA, 3177 bp, Artificial
  DNA sequence encoding a mature Chlamydomonas glycolate dehydrogenase (i.e. without transit peptide)
gctcgaggacctgcatcccctagctcgctagagcagcagacgcgccaggtcgctcaggttgctgttcagcagtcgactcagcaggcagtgaaggtcgttgtgccggccatcaaagtagacctggttggtgcggtcagctcggtgtctgagagcgacaaggtggagccgggtgtgttcaagaacgtggatggccaccgcttcgaggacggtcgctatgccgcttttgttgaggagattacaaagtttatccccaaggagcgccagtactcggaccccgtgcgcacattcgcgtatggcacggatgcctccttctaccggcttaacccgaagctggtagtgaaggtgcacaacgaggacgaggtccgccgcatcatgcccatcgcggagcggctgcaggtccctatcaccttccgcgcggccggcacgtcgctgtctgggcaggcaattaccgactcggtgctcattaagctgagccacacgggcaagaacttccgcaactttaccgtgcacggcgacggtagcgtgatcacggtggagccgggcctcattggcggcgaggtgaaccgcatcctggcggcacaccagaagaagaacaagctgcccatccagtacaagatcggacccgacccctcctccatcgacagctgcatgatcggcggcatcgtgtccaacaacagcagcggcatgtgctgcggcgtgagccagaacacctaccacacgctgaaggacatgcgggtggtgttcgtagacggaacggtgctggacacggccgaccccaactcgtgcaccgccttcatgaagagccaccgctcgctggtggatggcgtcgtgagcctggcgcgccgcgtgcaggccgacaaggagctgacggcgctcatccgccgcaagttcgccatcaagtgcaccaccggctactccctgaacgcgctggtggacttcccggtggacaaccccattgagatcatcaagcacctcatcatcggcagcgagggcacgctgggcttcgtcagccgcgccacctacaacaccgtgcccgagtggcccaacaaggcctcggccttcatcgtgttcccggacgtgcgcgccgcctgcaccggcgcctcggtgctgcgcaacgagacgtccgtggacgcggtggagctgtttgaccgcgccagcctgcgcgagtgcgagaacaacgaggacatgatgcgcctggtgcccgacatcaagggctgcgaccccatggcggcagcgctgctgatcgagtgccgcggccaggacgaggccgcactgcagagccgcattgaggaggtggtgcgcgtgctgacggcggcgggcctgcccttcggcgccaaggccgcgcagcccatggccatcgacgcctaccccttccaccacgaccagaagaacgccaaggtcttctgggacgtgcgcaggggcctgatccccattgtgggcgcggcgcgcgagcccggcacatccatgctgatcgaggacgtggcctgccccgtggacaagctggccgacatgatgatcgacctgatcgacatgttccagcgccacggctaccacgacgcctcctgcttcggccacgcgctcgagggcaaccttcacttggtgttctcgcagggcttccgcaacaaggaggaggtgcagcgcttcagcgacatgatggaggagatgtgccacctggtggccaccaagcactcgggcagcctcaagggcgagcacggcacgggccgcaacgtggcgccgttcgtggagatggagtggggcaacaaggcgtacgagctgatgtgggagctcaaggcgctgttcgaccccagccacaccctcaacccgggcgtcatcctcaaccgcgaccaggacgcgcacatcaagttcctgaagccctcgcccgcggcctcgcccatcgtcaaccgctgcatcgagtgcggcttctgcgagtccaactgcccctcgcgcgacatcacgctcacgccgcgccagcgcatctccgtgtaccgcgagatgtaccgcctcaagcagctgggcccgggcgccagcgaggaggagaagaagcagctggcggccatgagcagctcgtacgcctacgacggcgagcagacgtgcgcggcggacggcatgtgccaggagaagtgccccgtcaagatcaacacgggagacctgatcaagtcgatgcgtgccgagcacatgaaggaggagaagaccgccagcggcatggcagactggctggccgccaacttcggcgtcatcaactccaacgtgccgcgcttcctcaacatcgtcaacgccatgcacagcgtggtgggctcggcgcctctgtccgccatcagccgcgcgctcaacgccgccaccaaccacttcgtgccggtgtggaacccctacatgcccaagggcgcggcgccgctcaaggtgcccgccccgccggcgccggcagctgctgaggcctcgggcatcccgcgcaaggtggtgtacatgcccagctgcgtgacgcgcatgatgggccccgccgcctccgacaccgagacggcggcggtgcacgagaaggtgatgagcctgttcggcaaggccggctacgaggtgatcatccccgagggcgtggccagccagtgctgcggcatgatgttcaacagccgcggcttcaaggacgccgccgccagcaagggcgcggagctggaggcggcgctgctcaaggcctcggacaatggcaagatccccatcgtcatcgacacctcgccctgcctggcgcaggtgaagagccagatcagcgagccgtcgctgcgcttcgcgctgtacgagccggttgagttcatccggcacttcctggtggacaagctggagtggaagaaggtgcgcgaccaggtggccatccacgtgccctgctcctccaagaagatgggcatcgaggagtccttcgcgaagctggcgggcctgtgcgccaacgaggtggtgccctcgggcattccttgctgcggcatggcgggcgaccgcggcatgcgcttccccgagctgaccggcgcctcgctgcagcacctcaacctgcccaagacctgcaaggacggctactccaccagccgcacctgcgagatgtcgctcagcaaccacgccggcatcaacttcaggggcctggtgtacctggtggatgaggccacggcgcctaagaagcaggccgccgctgccaagaccgcgtaa

SEQ ID NO: 12 - protein, 1058 aa, Artificial
  Synthetic Construct
ARGPASPSSLEQQTRQVAQVAVQQSTQQAVKVVVPAIKVDLVGAVSSVSESDKVEPGVFKNVDGHRFEDGRYAAFVEEITKFIPKERQYSDPVRTFAYGTDASFYRLNPKLVVKVHNEDEVRRIMPIAERLQVPITFRAAGTSLSGQAITDSVLIKLSHTGKNFRNFTVHGDGSVITVEPGLIGGEVNRILAAHQKKNKLPIQYKIGPDPSSIDSCMIGGIVSNNSSGMCCGVSQNTYHTLKDMRVVFVDGTVLDTADPNSCTAFMKSHRSLVDGVVSLARRVQADKELTALIRRKFAIKCTTGYSLNALVDFPVDNPIEIIKHLIIGSEGTLGFVSRATYNTVPEWPNKASAFIVFPDVRAACTGASVLRNETSVDAVELFDRASLRECENNEDMMRLVPDIKGCDPMAAALLIECRGQDEAALQSRIEEVVRVLTAAGLPFGAKAAQPMAIDAYPFHHDQKNAKVFWDVRRGLIPIVGAAREPGTSMLIEDVACPVDKLADMMIDLIDMFQRHGYHDASCFGHALEGNLHLVFSQGFRNKEEVQRFSDMMEEMCHLVATKHSGSLKGEHGTGRNVAPFVEMEWGNKAYELMWELKALFDPSHTLNPGVILNRDQDAHIKFLKPSPAASPIVNRCIECGFCESNCPSRDITLTPRQRISVYREMYRLKQLGPGASEEEKKQLAAMSSSYAYDGEQTCAADGMCQEKCPVKINTGDLIKSMRAEHMKEEKTASGMADWLAANFGVINSNVPRFLNIVNAMHSVVGSAPLSAISRALNAATNHFVPVWNPYMPKGAAPLKVPAPPAPAAAEASGIPRKVVYMPSCVTRMMGPAASDTETAAVHEKVMSLFGKAGYEVIIPEGVASQCCGMMFNSRGFKDAAASKGAELEAALLKASDNGKIPIVIDTSPCLAQVKSQISEPSLRFALYEPVEFIRHFLVDKLEWKKVRDQVAIHVPCSSKKMGIEESFAKLAGLCANEVVPSGIPCCGMAGDRGMRFPELTGASLQHLNLPKTCKDGYSTSRTCEMSLSNHAGINFRGLVYLVDEATAPKKQAAAAKTA

SEQ ID NO: 13 - DNA, 1869 bp, artificial
  DNA sequence encoding a truncated Chlamydomonas glycolate dehydrogenase
gctcgaggacctgcatcccctagctcgctagagcagcagacgcgccaggtcgctcaggttgctgttcagcagtcgactcagcaggcagtgaaggtcgttgtgccggccatcaaagtagacctggttggtgcggtcagctcggtgtctgagagcgacaaggtggagccgggtgtgttcaagaacgtggatggccaccgcttcgaggacggtcgctatgccgcttttgttgaggagattacaaagtttatccccaaggagcgccagtactcggaccccgtgcgcacattcgcgtatggcacggatgcctccttctaccggcttaacccgaagctggtagtgaaggtgcacaacgaggacgaggtccgccgcatcatgcccatcgcggagcggctgcaggtccctatcaccttccgcgcggccggcacgtcgctgtctgggcaggcaattaccgactcggtgctcattaagctgagccacacgggcaagaacttccgcaactttaccgtgcacggcgacggtagcgtgatcacggtggagccgggcctcattggcggcgaggtgaaccgcatcctggcggcacaccagaagaagaacaagctgcccatccagtacaagatcggacccgacccctcctccatcgacagctgcatgatcggcggcatcgtgtccaacaacagcagcggcatgtgctgcggcgtgagccagaacacctaccacacgctgaaggacatgcgggtggtgttcgtagacggaacggtgctggacacggccgaccccaactcgtgcaccgccttcatgaagagccaccgctcgctggtggatggcgtcgtgagcctggcgcgccgcgtgcaggccgacaaggagctgacggcgctcatccgccgcaagttcgccatcaagtgcaccaccggctactccctgaacgcgctggtggacttcccggtggacaaccccattgagatcatcaagcacctcatcatcggcagcgagggcacgctgggcttcgtcagccgcgccacctacaacaccgtgcccgagtggcccaacaaggcctcggccttcatcgtgttcccggacgtgcgcgccgcctgcaccggcgcctcggtgctgcgcaacgagacgtccgtggacgcggtggagctgtttgaccgcgccagcctgcgcgagtgcgagaacaacgaggacatgatgcgcctggtgcccgacatcaagggctgcgaccccatggcggcagcgctgctgatcgagtgccgcggccaggacgaggccgcactgcagagccgcattgaggaggtggtgcgcgtgctgacggcggcgggcctgcccttcggcgccaaggccgcgcagcccatggccatcgacgcctaccccttccaccacgaccagaagaacgccaaggtcttctgggacgtgcgcaggggcctgatccccattgtgggcgcggcgcgcgagcccggcacatccatgctgatcgaggacgtggcctgccccgtggacaagctggccgacatgatgatcgacctgatcgacatgttccagcgccacggctaccacgacgcctcctgcttcggccacgcgctcgagggcaaccttcacttggtgttctcgcagggcttccgcaacaaggaggaggtgcagcgcttcagcgacatgatggaggagatgtgccacctggtggccaccaagcactcgggcagcctcaagggcgagcacggcacgggccgcaacgtggcgccgttcgtggagatggagtggggcaacaaggcgtacgagctgatgtgggagctcaaggcgctgttcgaccccagccacaccctcaacccgggcgtcatcctcaaccgcgaccaggacgcgcacatcaagttctaa

SEQ ID NO: 14 - protein, 622 aa, artificial
  Synthetic Construct
ARGPASPSSLEQQTRQVAQVAVQQSTQQAVKVVVPAIKVDLVGAVSSVSESDKVEPGVFKNVDGHRFEDGRYAAFVEEITKFIPKERQYSDPVRTFAYGTDASFYRLNPKLVVKVHNEDEVRRIMPIAERLQVPITFRAAGTSLSGQAITDSVLIKLSHTGKNFRNFTVHGDGSVITVEPGLIGGEVNRILAAHQKKNKLPIQYKIGPDPSSIDSCMIGGIVSNNSSGMCCGVSQNTYHTLKDMRVVFVDGTVLDTADPNSCTAFMKSHRSLVDGVVSLARRVQADKELTALIRRKFAIKCTTGYSLNALVDFPVDNPIEIIKHLIIGSEGTLGFVSRATYNTVPEWPNKASAFIVFPDVRAACTGASVLRNETSVDAVELFDRASLRECENNEDMMRLVPDIKGCDPMAAALLIECRGQDEAALQSRIEEVVRVLTAAGLPFGAKAAQPMAIDAYPFHHDQKNAKVFWDVRRGLIPIVGAAREPGTSMLIEDVACPVDKLADMMIDLIDMFQRHGYHDASCFGHALEGNLHLVFSQGFRNKEEVQRFSDMMEEMCHLVATKHSGSLKGEHGTGRNVAPFVEMEWGNKAYELMWELKALFDPSHTLNPGVILNRDQDAHIKF

SEQ ID NO: 15 - DNA, 1479 bp, Synechocystis sp.
  CDS (1)..(1479): Synechocystis GDH
atggccattttctcccccgtcaacgccgttaccgatattattccccagctcgaaaaaattgttggccaggatggagtaattaaacgcaaagacgagctattcacctacgaatgcgacggtttaacgggttatcgacaacggccggccctggtggttttgccccgcacaacggaacaggtagccacaatagtgaaactttgtcacgatcgccaaattccttggattgccaggggggctggcacagggttatcggggggagccttgccgggggccgatagcctattgattgtcaccactcgcatgcggcaaattttggcagtagattacgacaaccagaccattgttgtccagccgggggtggtgaataactgggttacccaaaccgttagtggggctggcttttactatgcccctgatccttccagtcagattgtctgctccattggcggtaatattgcggaaaattccggtggagttcattgtttgaaatatggcaccaccaccaaccatgtgctgggcttgaaactggttattcccgatggctccattgtggaagtaggggggcaagtccccgaaacgccgggctacgatttaaccggtttatttgttggttccgaaggaaccctaggcatcgccacagaaatcaccctaaaaattctcaaaaccccagaatctatctgtgtcgtattggcggattttctttctctcgaagccaccgcccaatccgtggccgatatcattgcggcgggcatcgtcccagcgggcatggaaattatggacaatttcagcatcaatgcggtggaagacgtggtggccaccaattgttaccccagggatgcggcggccattttgttagtggaactggacggtctgcccatcgaagtggaattaaaccaagccaaagtagaagaaatttgccgcaacaatggagcccgcaacacggcgatcgcctacgaccaagaaacccgcctaaaaatgtggaaaggaagaaaagcggcctttgcggcggcgggtaaactaagccccagttactttgtccaagatggtgtggtaccccggactcaattggtacagattttaagcgacattaatgatttaagtaagaaatatggctttgccattgccaatgttttccatgccggagacggtaatttacatcccctaattttgtatgatcaaaaagtaccaggagcctgggaaaaagtggaagaattggggggagaaatccttaaacgctgtgtggaattggggggaagtttatccggagaacacggcattggcattgataaaaattgctttatgcccaatatgttcaacgaagtagatttagaaacaatgcaatgggtcagacaatgttttaatcctgataacttagctaatcctggtaagctttttcctaccccccgcagttgtggagaagtggccaatgcccaacggcttaacctaggccaggacaagaaaatggaggaaatttattga

SEQ ID NO: 16 - protein, 492 aa, Synechocystis sp.
MAIFSPVNAVTDIIPQLEKIVGQDGVIKRKDELFTYECDGLTGYRQRPALVVLPRTTEQVATIVKLCHDRQIPWIARGAGTGLSGGALPGADSLLIVTTRMRQILAVDYDNQTIVVQPGVVNNWVTQTVSGAGFYYAPDPSSQIVCSIGGNIAENSGGVHCLKYGTTTNHVLGLKLVIPDGSIVEVGGQVPETPGYDLTGLFVGSEGTLGIATEITLKILKTPESICVVLADFLSLEATAQSVADIIAAGIVPAGMEIMDNFSINAVEDVVATNCYPRDAAAILLVELDGLPIEVELNQAKVEEICRNNGARNTAIAYDQETRLKMWKGRKAAFAAAGKLSPSYFVQDGVVPRTQLVQILSDINDLSKKYGFAIANVFHAGDGNLHPLILYDQKVPGAWEKVEELGGEILKRCVELGGSLSGEHGIGIDKNCFMPNMFNEVDLETMQWVRQCFNPDNLANPGKLFPTPRSCGEVANAQRLNLGQDKKMEEIY